(19) United States
(12) Patent Application Publication
(10) Pub. No.: US 2008/0254487 A1
(43) Pub. Date: Oct. 16, 2008

(54) Connective Tissue Growth Factor Signaling

(75) Inventors: Stephen J. Klaus, San Francisco, CA (US); David Y. Liu, Palo Alto, CA (US); Irina Aizman, Cupertino, CA (US)

Correspondence Address: FIBROGEN, INC.;INTELLECTUAL PROPERTY DEPARTMENT, 225 GATEWAY BOULEVARD, SOUTH SAN FRANCISCO, CA, 94080, US

(73) Assignee: FIBROGEN, INC., South San Francisco, CA (US)

(21) Appl. No.: 10/586,318

(22) PCT Filed: Jan. 14, 2005
(86) PCT No.: PCT/US2005/001275

§ 371 Date: Jun. 23, 2008

Related U.S. Application Data
(60) Provisional application No. 60/537,053, filed on Jan. 16, 2004.

(51) Int. Cl. G01N 33/566; C07H 3/00; C12Q 1/02

(52) U.S. Cl. 435/7.21; 536/123.1; 435/29; 436/501

(57) ABSTRACT

The present invention provides compounds and agents that modulate CTGF-mediated cell adhesion and/or binding of CTGF to cells. The invention further provides assays that may be used to identify additional modulators of CTGF-mediated cell adhesion and CTGF binding to cells, and assays that may be used to identify compounds or agents that modulate interaction of CTGF with HSPGs.

This application claims the benefit of U.S. Provisional Application Ser. No. 60/537,053, filed on 16 Jan. 2004, incorporated by reference herein in its entirety.

### FIELD OF THE INVENTION

The present invention relates to growth factor signaling, and in particular modulation of connective tissue growth factor signaling. The invention provides compounds and methods to modulate CTGF-mediated cell adhesion and CTGF binding to cells, thereby modulating CTGF signaling. The invention further provides assays used to identify additional modulators of CTGF-mediated cell adhesion and CTGF binding to cells.

### BACKGROUND OF THE INVENTION

Connective Tissue Growth Factor (CTGF) is a 36 kD, cysteine-rich, heparin-binding, secreted glycoprotein with demonstrated effects in various physiological and pathological contexts. CTGF promotes cell proliferation, migration, adhesion, and tube formation of vascular endothelial cells; growth and migration of vascular smooth muscle cells; and proliferation, adhesion, and matrix production of fibroblasts. (Grotendorst and Bradham, U.S. Pat. No. 5,408,040; Babic et al. (1999) Mol Cell Biol 19:2958-2966; Shimo et al. (1999) J Biochem (Tokyo) 126:137-145; Fan et al. (2000) Eur J Cell Biol 79:915-923; Chen et al. (2001) J Biol Chem 276:10443-10452; and Frazier et al. (1996) J Invest Dermatol 107:404-411.) CTGF has been implicated in a number of disorders and conditions, including, but not limited to, disorders involving angiogenesis, fibrosis, and other conditions with proliferative aspects such as tumor formation and growth. (See, e.g., International Publication No. WO 96/38172.)

CTGF expression is induced by a variety of factors including members of the TGFβ superfamily, which includes TGFβ-1, -2, and -3, bone morphogenetic protein (BMP)-2, and activin; dexamethasone, thrombin, vascular endothelial growth factor (VEGF), and angiotensin II. (Franklin (1997) Int J Biochem Cell Biol 29:79-89; Wunderlich (2000) Graefes Arch Clin Exp Opthalmol 238:910-915; Denton and Abraham (2001) Curr Opin Rheumatol 13:505-511; and Riewald (2001) Blood 97:3109-3116.) Although CTGF has been shown to interact with numerous factors including VEGF, TGFβ, insulin-like growth factor (IGF), integrins, and heparan sulfate proteoglycans (HSPGs), the physiological importance of such interactions is not fully understood. (Inoki et al. (2002) FASEB J 16: 219-221; Abreu et al. (2002) Nat Cell Biol 4: 599-604; Kim et al. (1997) Proc Natl Acad Sci USA 94:12981-12986; Lau and Lam (1999) Exp Cell Res 248:44-57; Gao and Brigstock (2004) J Biol Chem 279:8848-8855.)

Association of CTGF with cells is dependent on heparan sulfate moieties on the cell surface. Both CTGF-mediated cell adhesion and CTGF signaling are abrogated by heparinase treatment of cells or inclusion of soluble heparin. (See, e.g., Gao and Brigstock (2003) Hepatol Res 27:214-220; Gao and Brigstock (2003) J Biol Chem 10.1074/jbc.M313204200; and Nishida et al. (2003) J Cell Physiol 196(2):265-275.) Further, CTGF is liberated from cells and or cell-associated matrices upon addition of soluble heparin. (See, e.g., Riser et al. (2000) J Am Soc Nephrol 11:25-38.) Various heparan sulfate proteoglycans (HSPGs), including low density lipoprotein receptor-related protein (LRP) and perlecan, have been implicated in CTGF binding and signaling. (See, e.g., Segarini et al. (2001) J Biol Chem 276(44):40659-40667; Gao and Brigstock (2003) Hepatol Res 27:214-220; and Nishida et al. (2003) J Cell Physiol 196(2):265-275.) Heparan sulfate binding by CTGF and other CCN family members, e.g., Cyr61, is also important for interaction with other receptors, such as integrins. (See, e.g., Chen et al. (2001) J Biol Chem 276:10443-100452; and Gao and Brigstock (2003) J Biol Chem 10.1074/jbc.M313204200.)

### Heparan Sulfate Proteoglycans

HSPGs are components of the extracellular milieu and are classified as either membrane anchored, e.g., glypicans; transmembrane, e.g., syndicans; or cell associated, e.g., perlecan. Additionally, HSPGs include cell membrane proteins such as betaglycan, CD44/epican, and testican. HSPGs consist of a core protein decorated with covalently linked heparan sulfate (HS) chains. (See, e.g., Bernfield et al. (1999) Annu Rev Biochem 68:729-777.) The HS chains are polysaccharides composed of repeating disaccharide units of uronic acid (iduronate or glucuronate) and glucosamine. (Bernfield et al., supra.) The disaccharide units are selectively acetylated at the N position of glucosamine; sulfated at the N, 3-O, and 6-O positions of glucosamine; and/or sulfated at the 2-O position of iduronic acid residues.

HSPGs mediate signaling activities based on the structure and sulfation of their HS chains, which influence interaction with signaling molecules. (See, e.g., Rapraeger (2002) Methods Cell Biol 69:83-109.) For example, specific sulfation of 2-O and 6-O positions on HS chains is necessary for fibroblast growth factor (FGF) signal transduction. Specifically, the 2-O sulfation is required for binding of basic FGF to heparin, and 6-O sulfation is required for bFGF dimerization and receptor activation. (Pye et al. (2000) Glycobiology 10:1183-1192; Schlessinger et al. (2000) Mol Cell 6:743-750.) Additional signaling pathways that require HSPGs include Wnt, interferon (IFN)-γ, transforming growth factor (TGF)-β, vascular endothelial growth factor (VEGF), platelet-derived growth factor (PDGF), and hepatocyte growth factor. (Reichsman et al. (1996) J. Cell Biol. 135:819-827; Lortat-Jacob et al. (1995) Biochem J 310:497-505; Lyon et al. (1997) J Biol Chem 272:18000-18006; Soker et al. (1994) Biochem Biophys Res Commun 203:1339-1347; and Zioncheck et al. (1995) J Biol Chem 270:16871-16878.)

Sulfation of HS chains is tissue specific, and changes in sulfation have been correlated with regulatory changes in growth factor signaling. (See, e.g., Brickman et al. (1998) J Biol Chem 273:4350-4359; Ai et al. (2003) J Cell Biol 162:341-351.) Mutations that alter HSPG formation, organization, or sulfation lead to defects in signaling pathways. (See, e.g., Forsberg and Kjellen (2001) J Clin Invest 108:175-180; Takei et al. (2004) Development 131:73-82.) Similarly, mutations in enzymes that alter sulfation patterns on HSPGs at the cell surface can lead to modification in cell signaling. (See, e.g., Ai et al., supra.)

As HSPGs are required for binding of CTGF to cells and/or cell-associated matrices, and for CTGF signaling, compounds and agents that affect interaction between HSPGs and CTGF would be advantageous for modulating CTGF activities. The present invention provides compounds that modulate CTGF-mediated cell adhesion and CTGF binding to cells. The invention further provides methods of using the compounds to treat various disorders associated with CTGF. The invention further provides assays that can be used to identify additional modulators of CTGF-mediated cell adhesion and CTGF binding to cells.

### SUMMARY OF THE INVENTION

The present invention provides compounds and agents for modulating CTGF activities. In one aspect, the invention provides a compound or agent for modulating CTGF-mediated cell adhesion, wherein the compound, when added to a substrate comprising CTGF, modulates binding of cells to the substrate. In one embodiment, the CTGF is directly adsorbed to the substrate. In another embodiment, the CTGF is bound to a monoclonal antibody specific for CTGF, and the antibody is directly bound to the substrate. The antibody may be any antibody specific for a CTGF epitope. In a particular embodiment, the antibody specifically binds to an epitope contained within a region of human CTGF from amino acid 1 to 247 or to an orthologous region of a CTGF from another species.

In another aspect, the invention provides a compound or agent for modulating binding of CTGF to a cell, wherein the compound, when added to a cell, modulates binding of CTGF to the cell.

The CTGF for use in the various aspects and embodiments described above may be any CTGF, including a CTGF selected from the group consisting of endogenous CTGF, recombinant CTGF, and fragments of CTGF. Although any fragment of CTGF that retains the appropriate characteristics and activity required for cell adhesion and/or binding to cells can be used in these aspects and embodiments, CTGF fragments comprising at least amino acid 247 to 349 of human CTGF or an orthologous region of a CTGF from another species are specifically embodied.

The cell for use in the various aspects and embodiments described above may be any cell capable of CTGF-mediated adhesion and/or binding of CTGF. In certain embodiments, the cell is selected from the group consisting of a fibroblast, an endothelial cell, a transformed cell, and a cancer cell, e.g., an osteosarcoma cell. In specific embodiments, the fibroblast is selected from the group consisting of human foreskin fibroblast and human lung fibroblast.

In one aspect, the compounds or agents of the invention are sulfated polysaccharides. In one embodiment, the polysaccharide comprises a series of saccharide subunits joined in a(1,4) and/or b(1,3) linkage. The saccharides can be any saccharide or derivative thereof, e.g., glucose, galactose, mannose, fucose, neuraminic N-acetyl acid (NeuNAc), N-acetyl glucosamine, N-acetyl galactosamine, and xylose; or modified saccharide, e.g., a uronic acid including, but not limited to, glucuronate, galacturonate, and iduronate. In one embodiment, the polysaccharide comprises one or more disaccharide units consisting of one sugar selected from the group consisting of N-galactosamine and N-glucosamine, and one sugar selected from the group consisting of iduronate, glucuronate, and galactose. When the polysaccharide consists of more than one disaccharide, the disaccharides may be identical, for example, repeating units of D-glucuronate-D-glucosamine; or the disaccharides may differ, for example, a mixture of D-glucuronate-D-glucosamine disaccharides and D-iduronate-D-glucosamine disaccharides. In various embodiments, the polysaccharide may be a glycosaminoglycan, e.g., selected from the group consisting of chondroitin, dermatan, and heparan. The polysaccharide may comprise any number of saccharide subunits, in any order, and combined by any linkage. In particular embodiments, the polysaccharide comprises at least 5 saccharide subunits, more particularly at least 10, and even more particularly at least 20. In a specific embodiment, the polysaccharide comprises about 10 to 50 saccharide subunits.

In the various aspects and embodiments described above, the polysaccharide may be selectively acetylated at the N position of any glucosamine and/or galactosamine; sulfated at the N, 3-O, and 6-O positions of glucosamine and/or galactosamine; and/or sulfated at any hydroxyl group, e.g., the 2-O position of iduronic acid residues. The degree of sulfation can vary, and in particular embodiments the polysaccharide contains at least 1.5 sulfate groups per disaccharide, and more particularly at least 2.0 sulfate groups per disaccharide. In a specific embodiment, the polysaccharide contains about 2.0 to 3.5 sulfate groups per disaccharide.

The present invention further provides use of any of the compounds or agents to modulate CTGF activity. In one embodiment, the compounds or agents are used to modulate CTGF-mediated cell adhesion in a subject. In another embodiment, the compounds and agents are used to modulate binding of CTGF to a cell in a subject. The subject may be any subject, and in particular embodiments the subject is selected from a cell, a tissue, and an organ. In such embodiments, the use is typically performed ex vivo. In other embodiments, the subject is an animal, particularly a mammal, and more particularly a human.

In various aspects, compounds and agents of the invention may be used to treat a subject having or at risk for having a CTGF-associated condition or disorder. The CTGF-associated disorder may be any disorder for which CTGF has been implicated, or for which CTGF expression has been correlated with disease severity. CTGF-associated conditions or disorders include, but are not limited to, disorders involving angiogenesis, atherosclerosis, glaucoma, proliferative vitreoretinopathy, etc.; cancer, including acute lymphoblastic leukemia, dermatofibromas, breast cancer, breast carcinoma, glioma and glioblastoma, rhabdomyosarcoma and fibrosarcoma, desmoplasia, angiolipoma, angioleiomyoma, desmoplastic cancers, and prostate, ovarian, colorectal, pancreatic, gastrointestinal, and liver cancer; other tumor growth and metastases; etc.; disorders exhibiting altered expression and deposition of extracellular matrix-associated proteins, e.g., fibrotic disorders; arthritis, retinopathies such as diabetic retinopathy; nephropathies such as diabetic nephropathy; cardiac, pulmonary, liver, and kidney fibrosis, and diseases associated with chronic inflammation and/or infection. In certain embodiments, the disorder is selected from the group consisting of fibrosis, metaplasia, and cancer. In a particular embodiment, the condition or disorder is idiopathic pulmonary fibrosis. In another particular embodiment, the condition or disorder is diabetic nephropathy.

In another aspect, the invention provides use of the compounds or agents to reduce the likelihood of developing a CTGF-associated disorder in a subject having a predisposition to develop such a disorder. A predisposition may include, e.g., hyperglycemia, hypertension, or obesity in the subject. Such disorders may occur, e.g., due to diabetes, obesity, etc., and include diabetic nephropathy, retinopathy, and cardiovascular disease. Additionally, a predisposition may be suspected due to an event, e.g., a myocardial infarction, surgery, peritoneal dialysis, chronic and acute transplant rejection, chemotherapy, radiation therapy, trauma, orthopedic or paralytic immobilization, congestive heart failure, pregnancy, or varicosities in the subject.

The invention also provides methods for identifying compounds or agents that modulate CTGF activities. In one aspect, the invention provides a method for identifying compounds or agents that modulate CTGF-mediated cell adhesion, the method comprising the steps of (a) adsorbing a monoclonal antibody specific for CTGF to a first and second substrate; (b) binding CTGF to the antibody on the first and second substrate; (c) adding cells to the first substrate under suitable conditions for cells to adhere to CTGF; (d) adding a compound or agent and cells to the second substrate under suitable conditions for cells to adhere to CTGF; and (e) comparing the number of cells adhered to CTGF on the first substrate and the number of cells adhered to CTGF on the second substrate, wherein a difference between the number of cells adhered to the first substrate compared to the second substrate is indicative of a compound or agent that modulates CTGF-mediated adhesion. In one embodiment, the monoclonal antibody binds to a CTGF epitope contained within a region of human CTGF from amino acid 1 to 247 or to an orthologous region of a CTGF from another species, and wherein the antibody is adsorbed to the substrate.

In another aspect, the invention provides a method for identifying compounds or agents that modulate binding of CTGF to a cell, the method comprising the steps of (a) culturing cells capable of producing endogenous CTGF in the presence of a compound or agent for a suitable period of time; (b) measuring the level of CTGF in the culture medium; and (c) comparing the amount of CTGF in the culture medium to the amount of CTGF in culture medium from cells cultured in the absence of compound for an identical period of time, wherein a difference between the amount of CTGF in culture media in the presence of compound or agent relative to in the absence of compound or agent is indicative of a compound or agent that modulates binding of CTGF to a cell.

In the aspects and embodiments of the methods provided above, the cell may be any cell capable of CTGF-mediated adhesion and/or binding CTGF. In certain embodiments, the cell is selected from the group consisting of a fibroblast, an endothelial cell, a transformed cell, and a cancer cell, e.g., an osteosarcoma cell. In specific embodiments, the fibroblast is selected from the group consisting of human foreskin fibroblast and human lung fibroblast. In some embodiments, the cell produces endogenous CTGF constitutively; in other embodiments, the cells are induced to express CTGF using an appropriate stimulant, e.g., TGF-β, VEGF, angiotensin, etc.

In another aspect, the invention provides a method for identifying compounds or agents that modulate interaction between CTGF and an HSPG, the method comprising the steps of (a) incubating CTGF and the HSPG in the presence of a compound or agent under conditions suitable for interaction between CTGF and the HSPG; (b) measuring the amount of HSPG interacting with CTGF; and (c) comparing the amount of HSPG interacting with CTGF in the presence of compound to the amount of HSPG interacting with CTGF in the absence of compound, wherein a difference between the amount of HSPG interacting with CTGF in the presence of compound or agent relative to in the absence of compound or agent is indicative of a compound or agent that modulates interaction between CTGF and the HSPG. In a particular embodiment, the HSPG is betaglycan. In another embodiment, the HSPG is LDL receptor-related protein (LRP).

In the various aspects and embodiments of the methods provide above, the CTGF may be any CTGF, including a CTGF selected from the group consisting of endogenous CTGF, recombinant CTGF, and fragments of CTGF. Although any fragment of CTGF that retains the appropriate characteristics and activity required for cell adhesion, binding to cells, and/or interacting with an HSPG can be used in these aspects and embodiments, CTGF fragments comprising at least amino acid 247 to 349 of human CTGF or an orthologous region of a CTGF from another species are specifically embodied.

These and other embodiments of the subject invention will readily occur to those of skill in the art in light of the disclosure herein, and all such embodiments are specifically contemplated.

### BRIEF DESCRIPTION OF THE DRAWINGS

FIGS. 1A, 1B, and 1C show dose-dependent adhesion of cells to CTGF presented by epitope-specific anti-CTGF monoclonal antibodies.

FIGS. 2A, 2B, 2C, and 2D show adhesion of cells to CTGF is dependent on the orientation of CTGF, as defined by epitope-specific anti-CTGF antibodies, and requires CTGF domain 4.

FIGS. 3A, 3B, and 3C show adhesion of cells to CTGF is dependent on heparan sulfate moieties associated with the adhering cells.

FIG. 4 shows binding of CTGF to cells is effectively competed by heparin derivatives containing specific sulfation patterns, but not by derivatives lacking such sulfation.

FIGS. 5A, 5B, and 5C show adhesion of cells to CTGF (FIG. 5A) and binding of CTGF to cells (FIGS. 5B and 5C) can be competed by heparin derivatives containing specific sulfation patterns, but not by derivatives lacking such sulfation.

FIGS. 6A and 6B show adhesion of cells to CTGF (FIG. 6A) and binding of CTGF to cells (FIG. 6B) can be competed by polysaccharides comprising at least about 14 saccharide subunits.

FIGS. 7A and 7B show adhesion of cells to CTGF (FIG. 7A) and binding of CTGF to cells (FIG. 7B) can be competed by various polysaccharide constructs that contain a sufficient degree of sulfation.

FIGS. 8A and 8B show betaglycan directly interacts with CTGF, and betaglycan, TGF-β, and CTGF form a ternary complex associated with cell signaling.

FIG. 9 shows CTGF interacts with basic FGF, and that bFGF and betaglycan compete for binding to CTGF.

### DESCRIPTION OF THE INVENTION

Before the present compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless context clearly dictates otherwise. Thus, for example, a reference to “a fragment” includes a plurality of such fragments, a reference to an “antibody” is a reference to one or more antibodies and to equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

### Invention

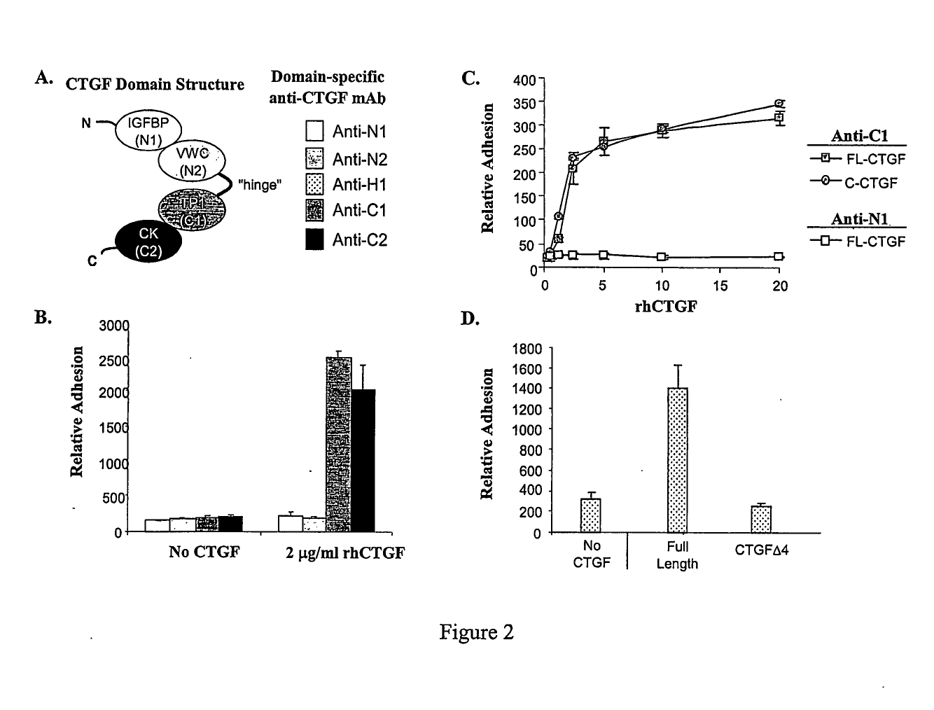

The present invention provides compounds or agents for modulating interaction between Connective Tissue Growth Factor (CTGF) and other cellular components. In one aspect, the compound or agent modulates CTGF-mediated cell adhesion. Such compounds or agents, when added to a substrate comprising CTGF, modulate cells concomitantly or subsequently added to the substrate from binding to the substrate. In one embodiment, the CTGF is directly adsorbed to the substrate. In another embodiment, the CTGF is bound to a monoclonal antibody specific for an epitope on CTGF, and the antibody is directly bound to the substrate. The epitope on CTGF to which the antibody binds is preferably a portion of human CTGF contained within amino acids 1 to 247 of human CTGF or to an orthologous region of a CTGF from another species, e.g., mouse FISP-12. In a particular embodiment, the antibody specifically binds to region C1 as shown in FIG. 2A.

In another aspect, the compound or agent modulates binding of CTGF to a cell. Such compounds or agents, when added to a cell, modulate binding of CTGF to the cell. In one embodiment, the compound or agent prevents exogenous CTGF from binding to the cell. In another embodiment, the compound or agent displaces CTGF bound to the cell. The CTGF bound to the cell may be endogenous CTGF or exogenous CTGF.

The CTGF adsorbed to substrate or used in cell binding may be recombinant CTGF, or CTGF isolated from various natural sources. The CTGF may be full-length CTGF, e.g., human CTGF (GenBank Accession No. P29279; Grotendorst and Bradham, U.S. Pat. No. 5,408,040), mouse FISP-12 (GenBank Accession No. AAA37627; Ryseck et al. (1991) Cell Growth Differ 2:225-233), rat CTGF (GenBank Accession No. AAD39132; Xu et al. (2000) J Cell Biochem 77:103-115), etc., or a fragment of CTGF. In one embodiment, the fragment of CTGF comprises at least amino acid 247 to 349 of human CTGF or an orthologous region from a CTGF from another species, e.g., mouse FISP-12.

In one aspect, the compounds or agents of the present invention are sulfated polysaccharides. In one embodiment, the polysaccharide comprises one or more disaccharide units consisting of one sugar selected from the group consisting of N-galactosamine and N-glucosamine, and one sugar selected from the group consisting of iduronate, glucuronate, and galactose. When the polysaccharide consists of more than one disaccharide, the disaccharides may be identical, for example, repeating units of D-glucuronate-D-glucosamine; or the disaccharides may differ, for example, a mixture of D-glucuronate-D-glucosamine disaccharides and D-iduronate-D-glucosamine disaccharides. In various embodiments, the polysaccharide may be a glycosaminoglycan, e.g., selected from the group consisting of chondroitin, dermatan, and heparan. In various aspects, the two sugar components of any disacharide may be joined in α(1,4) linkage or β(1,3) linkage; and may be selectively acetylated at the N position of glucosamine; sulfated at the N, 3-O, and 6-O positions of glucosamine; and/or sulfated at the 2-O position of iduronic acid residues.

In various embodiments, binding characteristics of any particular sulfated oligosaccharide can be modified by altering the length, e.g., the number of disacharide repeats, in the molecule. Such modifications can be measured for desired characteristics using binding and adhesion assays, as described in the Examples, using CTGF, fragments thereof, and other potential binding proteins, e.g., CK-containing proteins, as described below. In some embodiments, the polysaccharide comprises at least 10 saccharide subunits, e.g., 5 disaccharide repeats. In other embodiments, the polysaccharide comprises about 10 to 100 saccharide subunits, more particularly about 10 to 50 saccharide subunits. In a particular embodiment, the polysaccharide comprises about 18 saccharide subunits.

In various embodiments, binding characteristics of any particular sulfated oligosaccharide can be modified by altering the charge, e.g., the number of sulfated residues; and/or the charge distribution, e.g., the degree of N-sulfation, 2-O-sulfation, and 6-O-sulfation on respective sugar residues. Such modifications can be measured for desired characteristics using binding and adhesion assays, as described in the Examples, using CTGF, fragments thereof, and other potential binding proteins, e.g., CK-containing proteins, as described below. In some embodiments, the polysaccharide contains at least 1.5 sulfate groups per disaccharide. In other embodiments, the polysaccharide contains at least 2.0 sulfate groups per disaccharide. In a particular embodiment, the polysaccharide contains about 2.0 to 3.5 sulfate groups per disaccharide.

In some embodiments, the sulfated polysaccharides may be soluble molecules. Such soluble forms are useful as therapeutic agents for use in modulating the association of CTGF with cells, the extracellular matrix, or other components, e.g., growth factors, etc. In other embodiments, the sulfated polysaccharides may be attached to a peptide or protein; or to a solid or semi-solid matrix.

In one aspect, the compound or agent is a chondroitin sulfate, wherein the chondroitin sulfate contains at least 1.5 and particularly about 2.0 to 3.0 sulfate groups per disaccharide. Such compounds or agents are herein described as over-sulfated (OS) chondroitin sulfate. In another aspect, the compound or agent is a dermatan sulfate, wherein the dermatan sulfate contains at least 1.5 and particularly about 2.0 to 3.0 sulfate groups per disaccharide. Such compounds or agents are herein described as over-sulfated (OS) dermatan sulfate.

In another aspect, the compound or agent is a heparan sulfate, wherein the heparan sulfate contains about 2.0 to 3.0 sulfate groups per disaccharide. Such heparan sulfate moieties may be associated with protein, e.g., in the form of heparan sulfate proteoglycans (HSPGs), or attached to a solid matrix. In some embodiments, the heparan sulfate moieties may be soluble molecules, e.g., in a form chemically identical or similar to heparin.

The sulfated polysaccharide moieties encompassed in the present invention are generally defined according to their ability to bind CTGF or fragments thereof. Specific fragments of CTGF include the C-terminal half of CTGF, more specifically the domain encoded by exon 5. (See, e.g., International Publication Nos. WO 96/38172 and WO 00/35939.) Additionally, CTGF fragments for use in defining sulfated polysaccharide moieties of the present invention include those described in International Publication No. WO 99/07407; Gao and Brigstock (2003), supra; Ball et al. (2003) J Endocrinol 176:R1-7; Ball et al. (1998) Biol Reprod 59:828-835; and Brigstock et al. (1997) J Biol Chem 272:20275-20282; all of which are incorporated by reference herein in their entirety.

In certain aspects, a fragment of CTGF is characterized by the presence of the cystine-knot (CK) domain. Cystine-knot domains are found in various proteins including glycoprotein hormones and extracellular proteins. The C-terminal cystine knot-like domain (CTCK), found in CTGF and several other CCN family members, and other growth factors, e.g., TGFβ, nerve growth factor-(NGF), platelet-derived growth factor (PDGF), noggin, and gonadotropin, consists of 2 highly twisted antiparallel pairs of beta-strands containing three disulphide bonds. The domain is non-globular and little is conserved among these presumed homologs except for their cysteine residues. The CT and CTCK domains are predicted to form homodimers. Such proteins containing cystine-knot domains may be used to further characterize heparan sulfate (HS) and/or heparin-like molecules of the invention. Specific molecules may be selected based on selectivity in binding among the various CK-containing proteins; e.g., a molecule may be selected based on its binding to CTGF and other CCN family members, but not other growth factors such as TGF-β, basic FGF (bFGF), etc.; or a molecule may be selected based on its binding to CTGF, but not other CCN family members; etc.

The compounds and agents of the present invention can be utilized to modulate the bioactivity of CTGF. In particular embodiments, the molecule alters CTGF bioactivity by altering the ability of CTGF to interact with a cell surface or an endogenous extracellular matrix-associated HSPG. As other signaling pathways, e.g., bFGF signaling, are known to involve HSPG binding, the present invention specifically provides methods to inhibit the ability of CTGF to interact with HSPG without affecting the activity of other heparin binding growth factors. Such methods comprise administering compound or agent of the invention, e.g., a sulfated polysaccharide, to a subject. In these particular embodiments, the molecule is characterized by its ability to inhibit CTGF-mediated cell adhesion or cell binding without affecting the binding or signaling of other factors, e.g., other CCN family members and/or other growth factors such as VEGF or bFGF, as desired.

The compounds and agents can be additionally characterized by their ability to modulate binding between CTGF and specific HSPGs. In one embodiment, the present invention provides specific HSPGs, herein identified as CTGF-binding components, whose interaction with CTGF can be used to further characterize compounds and agents of the invention. For example, CTGF and a particular HSPG can be combined under conditions suitable for interaction between CTGF and the HSPG. Compounds or agents can be added, and an increase or decrease in interaction, e.g., binding, between CTGF and the HSPG in the presence of compound relative to interaction, e.g., binding, between CTGF and the HSPG in the absence of compound is indicative of a compound that modulates said interaction. Interaction between CTGF and HSPG can be measured by any technique known to those of skill in the art. A particular method exemplified herein is coimmunoprecipitation, wherein binding and sequestration of a first component, e.g., CTGF, by direct binding to a CTGF-specific antibody, results in sequestration of the second component, e.g., the HSPG. Addition of a compound or agent either increases or decreases the amount of the second component sequestered with the first component.

In one particular embodiment, the HSPG that specifically interacts with CTGF is betaglycan. As used herein, “betaglycan”, also known as “TGF-β type III receptor”, is selected from human betaglycan (GenBank Accession No. AAA67061) or an orthologous protein obtained from any other species. (See, e.g., GenBank Accession No. CAB64374; GenBank Accession No. AAC28564; and GenBank Accession No. AAA40813.) Additionally, betaglycan may comprise any fragment of a full-length betaglycan protein, and especially fragments of betaglycan described, e.g., in Lopez-Casillas et al. (1994) J Cell Biol 124(4):557-568; and Pepin et al. (1995) FEBS Lett 377: 368-372; both of which are incorporated by reference herein in their entirety. Further, betaglycan may comprise naturally-occurring or recombinant soluble betaglycan as described, e.g., in Zhang et al. (2001) Immunol Cell Biol 79:291-297; and Vilchis-Landeros et al. (2001) Biochem J 355:215-222, both of which are incorporated by reference herein in their entirety.

Betaglycan is a 349 amino acid transmembrane glycoprotein with a large extracellular region, which binds TGF-β, and a small cytoplasmic region. Betaglycan is considered an “accessory” receptor, since it appears to regulate the interaction of TGF-β with the signaling receptors, TGF-β type I receptor and TGF-β type II receptor, and thus regulate cell stimulation by TGF-β. (See, e.g., López-Casillas et al. (1993) Cell 73:1435-1444; Sankar et al. (1995) J Biol Chem 270:13567-13572; Lastres et al. (1996) J Cell Biol 133:1109-1121; and Sun and Chen (1997) J Biol Chem 272:25367-25372.) The extracellular domain of betaglycan contains heparan and chondroitin sulphate chains; however, it is thought to be the core protein that binds TGF-β isoforms.

The present invention provides methods to modulate growth factor activity mediated by CTGF. For example, the present examples demonstrate that CTGF and TGF-β form a physical complex with betaglycan. As betaglycan is required for proper cell stimulation by TGF-β, in particular embodiments the present invention provides methods to alter TGF-β signaling by inhibiting CTGF interaction with cell surface HSPGs. In certain embodiments, the HSPG is betaglycan.

Further, the present examples demonstrate a novel interaction between CTGF and bFGF, and interactions between CTGF and betaglycan are modulated in the presence of bFGF. In particular embodiments, the invention provides methods to modulate CTGF signaling in conjunction with or mediated by bFGF by blocking the capacity of CTGF to interact with cell surface HSPGs. In certain embodiments, the HSPG is betaglycan.

As described above, members of the CCN family share the domain on CTGF responsible for HSPG interaction. Although the specificity between individual members of the CCN family and respective HSPG moieties may vary, a certain degree of similarity would be expected. The invention, by providing means to identify and distinguish between sulfated polysaccharides, e.g., HS or heparin-like molecules, specific for CTGF, and HS or sulfated polysaccharides, e.g., heparin-like molecules generally active against CCN family binding, provides methods that can be used to modulate various CCN family signaling pathways. Therefore, in some embodiments, the invention provides methods to modulate the ability of CTGF to alter signaling by blocking the capacity of CCN family members to interact with cell surface proteoglycans, e.g., HSPGs. In particular embodiments, the method modulates signaling by Wnt, a developmental and oncogenic factor modulated by CCN family proteins, e.g., Wisp-3. In certain embodiments, the HSPG is associated with activity of the LDL receptor-related protein (LRP).

Recently, it has been demonstrated that betaglycan also binds and regulates the actions of other members of the TGF-β superfamily. For example, betaglycan forms a complex with the type II activin receptor. This complex then binds inhibin A and prevents formation of functional activin type I/II receptor complexes. (See, e.g., Lewis et al. (2000) Nature 404:411-414.) The interaction between inhibin and betaglycan also prevents bone morphogenetic protein (BMP), e.g., BMP-2, BMP-7, and BMP-9, signaling. (See, e.g., Wiater and Vale (2003) J Biol Chem 278:7934-7941.) As CTGF interacts with betaglycan and forms ternary complexes with betaglycan and TGF-β, CTGF may also regulate other facets of betaglycan function. In any case, modifying interactions between betaglycan and signaling factors, e.g., inhibin, using methods of the invention is specifically contemplated. In specific aspects, the invention provides methods to modulate the ability of CTGF to alter activin signaling by blocking the capacity of CTGF to interact with cell surface HSPGs. In other aspects, the invention provides methods to modulate the ability of CTGF to alter inhibin activity by blocking the capacity of CTGF to interact with cell surface HSPGs. In still other aspects, the invention provides methods to modulate the ability of CTGF to alter BMP signaling by blocking the capacity of CTGF to interact with cell surface HSPGs. In particular embodiments, the HSPG is betaglycan.

In all of the embodiments described above, it is a specific aspect of the invention that the degree of inhibition in CTGF binding can be regulated using specific compounds or agents such as sulfated polysaccharides, e.g., HS or heparin-like molecules. As CTGF has been implicated in pathways that may not involve heparan sulfate, it is envisioned that specific pathways may not be affected by the present procedures. For example, CTGF has been show to interact with other growth factors, e.g., VEGF and IGF. The present invention contemplates modulation of certain CTGF bioactivities, such as those associated with TGF-β signaling, by altering the ability of CTGF to interact with cell surface or extracellular matrix-associated HSPGs, without affecting or being affected by, e.g., VEGF and/or IGF signaling.

### Assays

Compounds and agents of the invention are defined by their ability to modulate CTGF-mediated cell adhesion and/or binding of CTGF to cells. Additional compounds or agents may be identified by adding the compound or agent to one of the following assays, and measuring the ability of the compound or agent to modulate the relevant parameter, i.e., CTGF-mediated cell adhesion or binding of CTGF to cells.

Methods for measuring cell adhesion mediated by CTGF are generally known to those skilled in the art. (See, e.g., Babic et al. (1999) Mol Cell Biol 19:2958-296; Ball et al. (2003) J Endocrinol 176:R1-7.) Such methods typically involve application of CTGF directly to a substrate. Unsaturated protein binding capacity on the substrate is blocked, e.g., with bovine serum albumin, and then cells are brought in contact with the substrate under conditions suitable for cell adhesion to the substrate. Additional factors, e.g., chelators such as EDTA, peptides, organic compounds, antibodies, etc., may be incubated with the cells prior to plating or added concurrently with the cells. Plates are incubated for a suitable length of time, e.g., 30 to 60 min, at a suitable temperature, e.g., 25 to 37° C., to allow cells to adhere; the substrate is then washed, and adherent cells are measured. Cell measurements may be made by any method known in the art; e.g., cells may be fixed with formalin, stained, e.g., with methylene blue, and quantified by dye extraction and measurement of absorbance, e.g., at 620 mm.

In a particular embodiment, the assay of the present invention attaches CTGF to the substrate indirectly using epitope-specific capture antibodies. Substrate, e.g., a MAXISORP plate (Nalge Nunc International, Rochester N.Y.), is coated with a monoclonal antibody specific for a CTGF epitope, preferably an epitope contained within a region defined by amino acids 1 to 246 of human CTGF or an orthologous region of a CTGF from a different species, e.g., mouse FISP-12. In a particular embodiment, the antibody binds specifically to a region defined by C1 as shown in FIG. 2A. CTGF, or fragments thereof, are then added to the antibody-coated substrate. In various embodiments, the CTGF may be rhCTGF or fragments thereof, particularly fragments comprising the epitope specific for the antibody and a region from amino acid 247 to 349 of human CTGF or an orthologous region from a CTGF from another species. Alternatively, the antibody may bind to a specific tag incorporated into a recombinant CTGF, e.g., a histidine tag. Appropriately modified rhCTGF is then added to the antibody-coated substrate. Unsaturated protein binding capacity on the substrate is blocked, e.g., with bovine serum albumin, and then cells are brought in contact with the substrate under conditions suitable for cell adhesion as described above. Cells for use in such assays may be any cell capable of CTGF-mediated adhesion, e.g., fibroblasts and endothelial cells. In various embodiments, the cells are selected from the group consisting of fibroblasts, endothelial cells, and transformed or cancer cells, e.g., osteosarcoma cells. In particular embodiments exemplified herein, the cells are human foreskin fibroblasts (HFF).

Methods for measuring binding of CTGF to cells are generally known to those skilled in the art. (See, e.g., Nishida (1998) Biochem Biophys Res Commun 247:905-909; Segarini et al. (2001) J Biol Chem 276:40659-40667.) Such methods typically involve labeling CTGF with a detectable moiety, e.g., a radioactive or fluorescent tag, applying the labeled CTGF to cells, washing the cells to remove unbound CTGF, and then measuring the amount of label that remains associated with the cells. Cells may be attached to a substrate, e.g., a tissue culture plate, or in suspension. Labeling cells in suspension allows analysis by flow cytometry, e.g. using fluorescently labeled CTGF and a fluorescent-activated cell sorter (FACS).

In a preferred method, cells are suspended in media containing CTGF under conditions suitable for binding of CTGF to cells. Cells may optionally be treated prior to or concurrently with CTGF exposure; for example, cells may be treated with enzymes that alter cell surface moieties, molecules that compete competitively or non-competitively with CTGF for binding to cells, etc. Following incubation to allow CTGF to bind to cells, cells are washed and then incubated with fluorescently-labeled anti-CTGF antibody. The level of CTGF binding is then measured as fluorescence, e.g., using a FACS apparatus.

Alternatively, binding of CTGF to cells can be measured using a CTGF displacement assay. CTGF may be constitutively produced by a cell or added exogenously and allowed to bind to cells. In particular embodiments, cells are induced to produce CTGF, e.g., by treating with a factor that stimulates CTGF production. Such factors include, but are not limited to, TGF-β, VEGF, angiotensin, endothelin, glucose, and mechanical stress. Cells, in suspension or attached to a substrate, are treated with compound or agent, and displacement of CTGF from the cell surface is measured. Various methods of detecting displaced CTGF are generally known to those of skill in the art, including SDS-PAGE, ELISA, and immunoprecipitation. Cells for use in such assays may be any cell capable of CTGF-mediated adhesion, e.g., fibroblasts and endothelial cells. In various embodiments, the cells are selected from the group consisting of fibroblasts, endothelial cells, and transformed or cancer cells, e.g., osteosarcoma cells. In particular embodiments exemplified herein, the cells are HFF, human lung fibroblasts (HLF), or MG63 osteosarcoma cells.

### Use of Compounds and Agents

In one aspect, the compounds and agents of the invention may be used to modulate CTGF-mediated adhesion in a subject. In another aspect, the compounds or agents may be used to modulate binding of CTGF to cells in a subject. In various embodiments, the subject may be a cell, tissue, or organ, and the use may be performed ex vivo. For example, an organ for transplant may be treated by the compound or agent to displace CTGF bound to the cells of the organ. Such a use may retard or reduce fibrosis and organ failure subsequent to implantation in a host. In other embodiments, the subject may be an animal, particularly a mammal, and more particularly a human.

The compounds and agents of the invention are especially useful in therapeutic applications, to prevent or treat CTGF-associated disorders in a subject. The phrase “CTGF-associated disorders” as used herein refers to conditions and diseases associated with abnormal or altered expression or activity of CTGF. Abnormal expression of CTGF has been associated with cell proliferative disorders, such as those caused by endothelial cell proliferation; cell migration; tumor-like growths; general tissue scarring; and various diseases characterized by inappropriate deposition of extracellular matrix.

CTGF-associated disorders include, but are not limited to, disorders involving angiogenesis and other processes which play a central role in conditions such as atherosclerosis, glaucoma, proliferative vitreoretinopathy, etc.; cancer, including acute lymphoblastic leukemia, dermatofibromas, breast cancer, breast carcinoma, glioma and glioblastoma, rhabdomyosarcoma and fibrosarcoma, desmoplasia, angiolipoma, angioleiomyoma, desmoplastic cancers, and prostate, ovarian, colorectal, pancreatic, gastrointestinal, and liver cancer; other tumor growth and metastases; etc.

Additionally, the compounds and agents of the invention are useful in therapeutic applications to prevent or treat CTGF-associated disorders involving fibrosis. In one aspect, the compounds or agents of the invention are administered to a subject to prevent or treat a CTGF-associated disorder including, but are not limited to, disorders exhibiting altered expression and deposition of extracellular matrix-associated proteins, e.g., fibrotic disorders. In various aspects, the fibrosis may be localized to a particular tissue, such as epithelial, endothelial, or connective tissue; or to an organ, such as kidney, lung, or liver. Fibrosis can also occur in the eye and joints. In other aspects, the fibrosis may be systemic and involve multiple organ and tissue systems. CTGF-associated disorders include, for example, atherosclerosis, arthritis, retinopathies such as diabetic retinopathy; nephropathies such as diabetic nephropathy; cardiac, pulmonary, liver, and kidney fibrosis, and diseases associated with chronic inflammation and/or infection.

In another aspect, the invention provides use of the compounds or agents to reduce the likelihood of developing a CTGF-associated disorder in a subject having a predisposition to develop such a disorder. A predisposition may include, e.g., hyperglycemia, hypertension, or obesity in the subject. Such disorders may occur, e.g., due to diabetes, obesity, etc., and include diabetic nephropathy, retinopathy, and cardiovascular disease. Additionally, a predisposition may be suspected due to an event, e.g., a myocardial infarction, surgery, peritoneal dialysis, chronic and acute transplant rejection, chemotherapy, radiation therapy, trauma, orthopedic or paralytic immobilization, congestive heart failure, pregnancy, or varicosities in the subject.

Compounds and agents may be used in the formulation of a medicament, wherein the compound or agent is combined with other materials, which may include, but are not limited to, carriers, excipients, and solvents. Pharmaceutically acceptable excipients are available in the art, and include those listed in various pharmacopoeias. (See, e.g., the U.S. Pharmacopeia (USP), Japanese Pharmacopoeia (JP), European Pharmacopoeia (EP), and British pharmacopeia (BP); the U.S. Food and Drug Administration (www.fda.gov) Center for Drug Evaluation and Research (CEDR) publications, e.g., Inactive Ingredient Guide (1996); Ash and Ash, Eds. (2002) Handbook of Pharmaceutical Additives, Synapse Information Resources, Inc., Endicott N.Y.; etc.) Additionally, the active compound or agent for purposes of the methods herein may be combined with one or more additional therapeutic agents.

These and other embodiments of the present invention will readily occur to those of ordinary skill in the art in view of the disclosure herein.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications are intended to fall within the scope of the appended claims.

### Example 1

### Production of Recombinant Human CTGF (rhCTGF)

A recombinant human CTGF baculovirus construct was produced as described in Segarini et al. (2001, J Biol Chem 276:40659-40667). Briefly, a CTGF cDNA comprising only the open reading frame was generated by PCR using DB60R32 (Bradham et al. (1991) J Cell Biol 114:1285-94) as template and the primers 5′ gctccgcccgcagtgggatccATGaccgccgcc 3′ and 5′ ggatccggatccTCAtgccatgtctccgta 3′, which add BamHI restriction enzyme sites to the ends of the amplified product. The native start and stop codons are indicated in capital letters.

The resulting amplified DNA fragment was digested with BamHI, purified by electrophoresis on an agarose gel, and subcloned directly into the BamHI site of the baculovirus PFASTBAC1 expression plasmid (Invitrogen Corp., Carlsbad Calif.). The sequence and orientation of the expression cassette was verified by DNA sequencing. The resulting CTGF expression cassette was then transferred to bacmid DNA by site-specific recombination in bacteria. This bacmid was then used to generate a fully recombinant CTGF baculovirus in Spodoptera frugiperda SF9 insect cells according to protocols supplied by the manufacturer (BAC-TO-BAC Expression System manual; Invitrogen). Expansion of recombinant baculovirus titers in Sf9 insect cells was performed using standard procedures known in the art.

Hi5 insect cells were adapted for suspension growth by serial passage of cells in shake flask culture accompanied by enrichment at each passage for separated cells. Suspension Hi5 cells were cultured in 1 L SF900II SFM media (Invitrogen) supplemented with 20 μg/ml gentamicin (Mediatech, Inc., Herndon Va.) and 1× lipid (Invitrogen) in disposable 2.8 L Fernbach culture flasks (Corning Inc., Acton Mass.) on a shaker platform at 110 rpm at 27° C. Once cells reached a density of 1.0-1.5×10⁶ cells/ml with a viability of >95%, they were infected with recombinant baculovirus at a multiplicity of infection (MOI) of 10. The cultures were then incubated at 27° C. for an additional 40 to 44 hours. The conditioned media, which contains rhCTGF, was collected, chilled on ice, and centrifuged at 5000×g. The supernatant was then passed through a 0.45 mm filter.

Four liters of conditioned media was loaded over a 5 ml HI-TRAP heparin column (Amersham Biosciences Corp., Piscataway N.J.) pre-equilibrated with 50 mM Tris (pH7.5), 150 mM NaCl. The column was washed with 10 column volumes of 350 mM NaCl, 50 mM Tris (pH 7.5). CTGF was eluted from the column with an increasing NaCl salt gradient. Eluted fractions were screened by SDS-PAGE, and those containing CTGF were pooled.

Heparin purified CTGF was diluted to a final conductivity of 5.7 mS with non-pyrogenic double-distilled water and the pH was adjusted to 8.0. A Q-SEPHAROSE strong anion exchange column (Amersham Biosciences) containing approximately 23 ml resin connected in tandem with a carboxymethyl (CM) POROS polystyrene column (Applied Biosystems) containing approximately 7 ml resin was utilized for endotoxin removal, and capture and elution of purified rhCTGF. Prior to the sample load, the tandem column was washed with 0.5 M NaOH, followed by 0.1 M NaOH, and finally equilibration buffer. The load sample was passed over the tandem column, the Q-Sepharose column was removed, and CTGF was eluted from the CM POROS column (Applied Biosystems) with an increasing 350 mM to 1200 mM NaCl gradient. The purity of the eluted fractions containing CTGF was evaluated by SDS-PAGE analysis before forming a final sample pool.

### Example 2

### Anti-CTGF Monoclonal Antibodies

### 2.1 Antibody Production

Fully human monoclonal antibodies to human CTGF were prepared using HUMAB mouse strains HCo7, HCo12 and HCo7+HCo12 (Medarex, Inc., Princeton N.J.). Mice were immunized by up to 10 intraperitoneal (IP) or subcutaneous (Sc) injections of 25-50 mg recombinant human CTGF in complete Freund's adjuvant over a 24 week period. The immune response was monitored by retroorbital bleeds. Plasma was screened by ELISA (as described below), and mice with sufficient titers of anti-CTGF immunoglobulin were used for fusions. Mice were boosted intravenously with antigen 3 and 2 days before sacrifice and removal of the spleen.

Single cell suspensions of splenic lymphocytes from immunized mice were fused to one-fourth the number of P3×63-Ag8.653 nonsecreting mouse myeloma cells (American Type Culture Collection (ATCC), Manassas Va.) with 50% PEG (Sigma, St. Louis Mo.). Cells were plated at approximately 1×10⁵ cells/well in flat bottom microtiter plate and incubated for about two weeks in high-glucose DMEM (Mediatech, Herndon Va.) containing L-glutamine and sodium pyruvate, 10% fetal bovine serum, 10% P388D1 (ATCC) conditioned medium, 3-5% ORIGEN hybridoma cloning factor (Igen International, Gaithersburg Md.), 5 mM HEPES, 0.055 mM 2-mercaptoethanol, 50 mg/ml gentamycin, and 1× HAT (Sigma). After 1-2 weeks, cells were cultured in medium in which the HAT was replaced with HT. Individual wells were then screened by ELISA (described below). Antibody secreting hybridomas were replated, screened again, and, if still positive for anti-CTGF antibodies, were subcloned at least twice by limiting dilution. The stable subclones were then cultured in vitro to generate small amounts of antibody in tissue culture medium for characterization. One clone from each hybridoma that retained the reactivity of the parent cells was used to generate 5-10 vial cell banks stored in liquid nitrogen.

ELISA assays were performed as described by Fishwild et al. (1996, Nature Biotech 14:845-851). Briefly, microtiter plates were coated with 1-2 μg/ml purified recombinant CTGF in PBS at 50 μl/well, incubated at 4° C. overnight, then blocked with 20 μl/well 5% chicken serum in PBS/Tween (0.05%). Dilutions of plasma from CTGF-immunized mice or hybridoma culture supernatants were added to each well and incubated for 1-2 hours at ambient temperature. The plates were washed with PBS/Tween and then incubated with a goat-anti-human IgG Fₑ polyclonal antibody conjugated with horseradish peroxidase (HRP) for 1 hour at room temperature. After washing, the plates were developed with 0.22 mg/ml ABTS substrate (Sigma) and analyzed by spectrophotometer at 415-495 nm.

### 2.2 Antibody Characterization

Epitope mapping of antibodies by competitive binding experiments is well known by those skilled in the field of immunology. (See, e.g., Van Der Geld et al. (1999) Clinical and Experimental Immunology 118:487-96.) Each antibody population isolated from cells propagated from a unique cloned hybridoma cell was mapped and assigned to a specific binding domain on human CTGF using standard binding and blocking experiments. (See, e.g., Antibodies: A Laboratory Manual (1988) Harlow and Lane (eds), Cold Spring Harbor Laboratory Press; Tietz Textbook of Clinical Chemistry, 2nd ed., (1994) Chapter 10 (Immunochemical Techniques), Saunders; and Clinical Chemistry: Theory, Analysis, Correlation (1984) Chapter 10 (Immunochemical Techniques) and Chapter 11 (Competitive Binding Assays), C.V. Mosby, St. Louis.) For example, epitope mapping was performed by ELISA analysis using specific recombinantly expressed fragments of CTGF. Antibodies that recognized epitopes, e.g., on the N-terminal domain of CTGF were identified by ELISA analysis against immobilized fragments obtained from recombinant expression of exon 2 and/or exon 3 of the CTGF gene. Antibodies that specifically recognize N-terminal domains or N-terminal fragments of CTGF (e.g., anti-N1, an antibody having specificity for an N-terminal fragment epitope containing CTGF domain 1; anti-N2, an antibody having specificity for an N-terminal fragment epitope containing CTGF domain 2; etc.) or C-terminal domains or C-terminal fragments of CTGF (e.g., anti-C1, an antibody having specificity for a C-terminal fragment epitope containing CTGF domain 3; anti-C2, an antibody having specificity for a C-terminal fragment epitope containing CTGF domain 4; etc.) were selected and utilized in the following examples.

### Example 3

### Assays

### 3.1 Cell Adhesion Assay

Methods for measuring cell adhesion mediated by CTGF are generally known to those skilled in the art. (See, e.g., Babic et al. (1999) Mol Cell Biol 19:2958-296; Ball et al. (2003) J Endocrinol 176:R1-7.) In some experiments, wells of a MAXISORP plate (Nunc Nalgene) were treated with 10 μg/ml recombinant human CTGF (rhCTGF) to directly adsorb CTGF to the well. Alternatively, wells were coated with a human monoclonal antibody specific for human CTGF, and then were blocked with bovine serum albumin to prevent non-specific binding. 2 μg/1 ml rhCTGF or fragments thereof, or a vehicle control was added to each well. Plates were then washed 3 times with PBS, cells were added at a seed density of approximately 8×10³ cells/well, and plates were incubated for 45 minutes at 37° C. Wells were then washed twice, and the number of cells retained in each well was measured using a CYQUANT cell proliferation assay kit (Molecular Probes, Inc., Eugene Oreg.). Alternatively, attached cells were lysed in 2% Triton and lactate dehydrogenase (LDH) activity was measured using a cytotoxicity detection (LDH) kit (Roche Diagnostics Corp., Chicago, Ill.). LDH levels were compared against a standard curve generated using known numbers of cells, and results of experiment were expressed as numbers of attached cells per well.

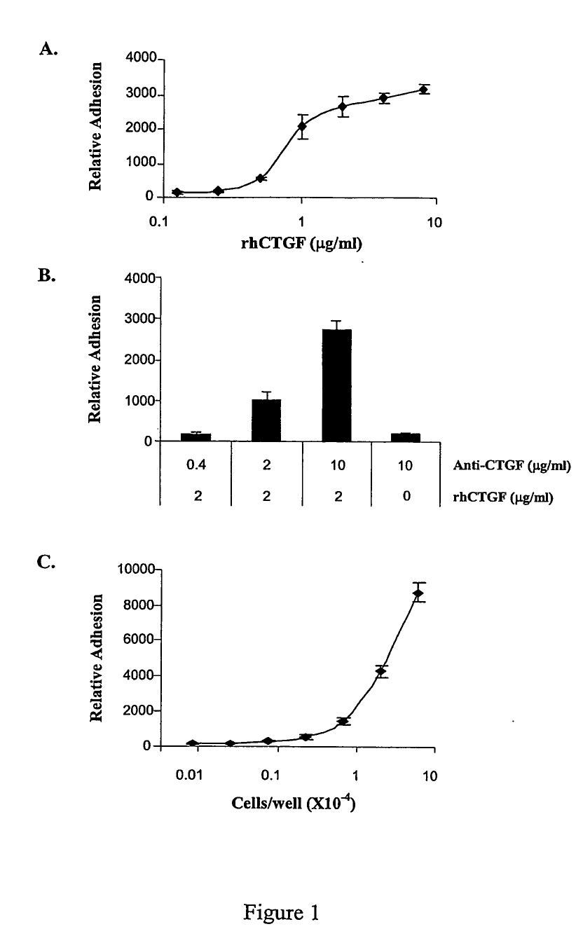

In experiments using human dermal foreskin fibroblast cells and a human monoclonal antibody specific for human CTGF domain 3 (anti-C1), dose-sensitive cell adhesion was seen when any of the parameters, i.e., amount of CTGF, anti-CTGF antibody, or cell number, was altered while the remaining parameters were held constant. For example, a dose-sensitive increase in the number of cells retained in each well was seen when either antibody concentration was held constant (10 μg/ml) and CTGF concentration was increased (FIG. 1A), or when CTGF concentration was held constant (2 μg/ml) and anti-CTGF antibody concentration was increased (FIG. 1B). Similarly, a dose-sensitive increase in the number of cells retained in each well was seen when cells were titrated in wells coated with a constant amount of antibody (10 μg/ml) and CTGF (2 μg/ml) (FIG. 1C).

### 3.2 CTGF Binding Assay

Methods for measuring binding of CTGF to cells are generally known to those skilled in the art. (See, e.g., Nishida (1998) Biochem Biophys Res Commun 247:905-909; Segarini et al. (2001) J Biol Chem 276:40659-40667.) In one method used herein, cells were suspended in media containing CTGF under conditions suitable for binding of CTGF to cellular targets, e.g., incubation at 4° C. Cells may optionally be treated prior to or concurrently with CTGF exposure; for example, cells may be treated with enzymes that alter cell surface moieties, molecules that compete competitively or non-competitively with CTGF for binding to cells, etc. Following incubation, cells were washed and then incubated with fluorescently-labeled anti-CTGF antibody. The level of CTGF binding was then measured using a FACS apparatus.

### 3.3 CTGF Displacement Assay

Alternatively, cell binding was measured using a CTGF displacement assay. The displacement assay used in the present examples measured displacement of CTGF endogenously produced by cells. HLF and MG63 cells were separately used in the displacement assay as follows. In all experiments, cells were plated in 96-well tissue culture plates at a seed density of 2.5×10⁴ cells/well. Cells were incubated for approximately 24 hours at 37° C., and then medium was replaced with a serum-free medium containing titrated amounts of sulfated polysaccharides (glycosaminoglycans, GAGs). In some experiments, constitutive CTGF expression accounted for the displaced CTGF, whereas in other experiments additional levels of CTGF were induced by addition of TGF-β2 in culture media 30 minutes prior to change of media and addition of GAGs. MG63 cells were incubated for either 3 or 6 hrs, and HLF cells were incubated for 20 hrs, at 37° C., and then conditioned media was assayed for N-fragment and full-length CTGF using a sandwich ELISA assay. (See International Publication No. WO 03/024308.)

### 3.4 Co-Immunoprecipitation Assay

Co-immunoprecipitation is a purification procedure used to determine if two different molecules, e.g., proteins, directly interact. Basically, an antibody specific to a protein of interest is added to a cell lysis under conditions suitable for antibody binding to the protein. The antibody-protein complex is then collected, e.g., using protein-G sepharose, which binds most antibodies. Any molecules that are bound to the precipitated protein will also be collected. Identification of proteins can be determined by, e.g., western blot or by direct sequencing of the purified protein(s). Several commercial kits, e.g., the PROFOUND co-immunoprecipitation kit from Pierce Biotechnology, Inc. (Rockford Ill.) are also available.

In the present examples, co-immunoprecipitations were performed as follows. The surface of intact cells was iodinated with ¹²⁵I prior to lysing cells and fractionating on a CTGF affinity column. Alternatively, CTGF and labeled cells were incubated for a period sufficient for CTGF binding to cells, and then cells were lysed and immunoprecipitations were performed using anti-CTGF specific antibodies. Antibody complexes were collected from the lysate using protein-G sepharose, and pelleted by centrifugation. Proteins eluted from affinity columns or collected by immunoprecipitation were analyzed by fractionation on SDS-PAGE and visualized by autoradiography. In similar experiments, unlabeled cells or specific proteins were mixed with CTGF alone or in the presence of additional factors, and immunoprecipitations were performed. Following fractionation, proteins were transferred to membranes and probed by western analysis.

### Example 4

### Regions of CTGF Involved in Cell Binding and Adhesion

### 4.1 Various Cell Types Utilize a Similar Mechanism in CTGF-Mediated Adhesion

The cell adhesion assay described in Example 3.1 was used to identify regions of CTGF involved in cell adhesion. Wells of a tissue culture plate were coated with monoclonal antibodies specific for either amino-terminal domains (anti-N1 or anti-N2 antibodies) or carboxy-terminal domains (anti-C1, -C2) of CTGF (see FIG. 2A). HFF were seeded into wells and adhesion was measured as described in Example 3.1.

As shown in FIG. 2B, antibodies specific for epitopes associated with the C-terminal domain of CTGF presented CTGF to cells in a manner that facilitated cell adhesion. However, antibodies specific for epitopes on the N-terminal domain of CTGF did not orient CTGF in a manner that allowed cell adhesion.

### 4.2 The C-Terminal Half of CTGF Mediates Cell Adhesion

To further define the region of CTGF responsible for cell adhesion, the procedure used in Example 4.1 was further modified as follows. Wells of a tissue culture plate were coated with monoclonal antibodies specific for either amino-terminal (anti-N1) or carboxy-terminal (anti-C1) domains of CTGF (see FIG. 2A). Wells were then seeded with HFF in the presence of no CTGF, full-length CTGF, the N-terminal half of CTGF (NH2 fragment), or the C-terminal half of CTGF (COOH fragment). Wells were incubated and adhesion was measured as described in Example 3.1.

Consistent with the results shown in Example 4.1, presentation of full-length CTGF using anti-C1 antibodies facilitated cell adhesion, whereas presentation using anti-N1 antibodies did not (FIG. 2C). Further, the C-terminal half of CTGF, when captured using anti-C1 antibodies, was sufficient to provide cell adhesion equivalent to adhesion provided by full-length CTGF. Additionally, the binding was dose responsive, increasing with increasing amounts of CTGF or CTGF fragment. The N-terminal half of CTGF, however, did not provide a suitable substrate for cell adhesion (FIG. 2C). The data show that the C-terminal half of CTGF mediates CTGF-dependent adhesion.

### 4.3 CTGF-Dependent Adhesion Requires Domain 4

The cell adhesion assay described in Example 3.1 was used to further define the portion of the C-terminal half of CTGF involved in cell adhesion. Wells of a tissue culture plate were coated with monoclonal antibodies specific for the “hinge” domain of CTGF (anti-H1 antibodies) (see FIG. 2A). Wells were then seeded with HFF (8×10⁴ cells/well) in the presence of no CTGF, full-length CTGF, or a CTGF construct lacking domain 4 (CTGFΔ4). Wells were incubated and adhesion was measured as described in Example 3.1.

Although HFF were able to adhere to full-length CTGF, they were not able to bind to CTGF lacking domain 4 (CTGFΔ4) (FIG. 2D). This result suggests that domain 4, which contains the cystine knot (CK) motif, is necessary for CTGF-mediated cell adhesion.

### Example 5

### HSPGs are Required for CTGF Binding and CTGF-Mediated Adhesion

The following example utilized various sulfated polysaccharides as characterized in Table 1. Cell adhesion and cell binding assays were conducted as described in Examples 3.1, 3.2, and 3.3.

TABLE 1
Sulfated polysaccharides

| | | Degree of sulfation |
|---|---|---|
| Sulfated Polysaccharide* | Avg Size | (sulfates/disaccharide) |
| Heparin | 16K | 2.4 |
| Heparin VI | 15K | 2-2.4 |
| OS-heparin | 12K | 3.5-4 |
| heparan sulfate | 6-9K | 0.5-1 |
| OS-heparan sulfate | 7.5K | 3-3.5 |
| keratan sulfate | 8K | 1 |
| chondroitin 4-sulfate (A) | 50K | 0.5-1 |
| OS-chondroitin 4-sulfate (A) | 40K | 2-3 |
| chondroitin 6-sulfate (C) | 50-100K | 1 |
| dermatan sulfate (CS-B) | 16-25K | 0.7-1.2 |
| OS-dermatan sulfate (CS-B) | 25K | 2.5-3 |
| Heparin Polysaccharide IV | 12K | 2-2.5 |
| Heparin Polysaccharide II | 7K | 2-2.5 |
| Heparin Oligosaccharide II | 4.2K | 2 |
| Heparin Decasaccharide | 3K | 1.8-2 |
| Sulodexide | 8K | |

*obtained from Sigma-Aldrich, St. Louis MO; Neoparin, Inc., San Leandro, CA; and Celsus Laboratory, Cincinnati OH

### 5.1 Heparan Sulfate is Involved in CTGF Binding and CTGF-Dependent Cell Adhesion

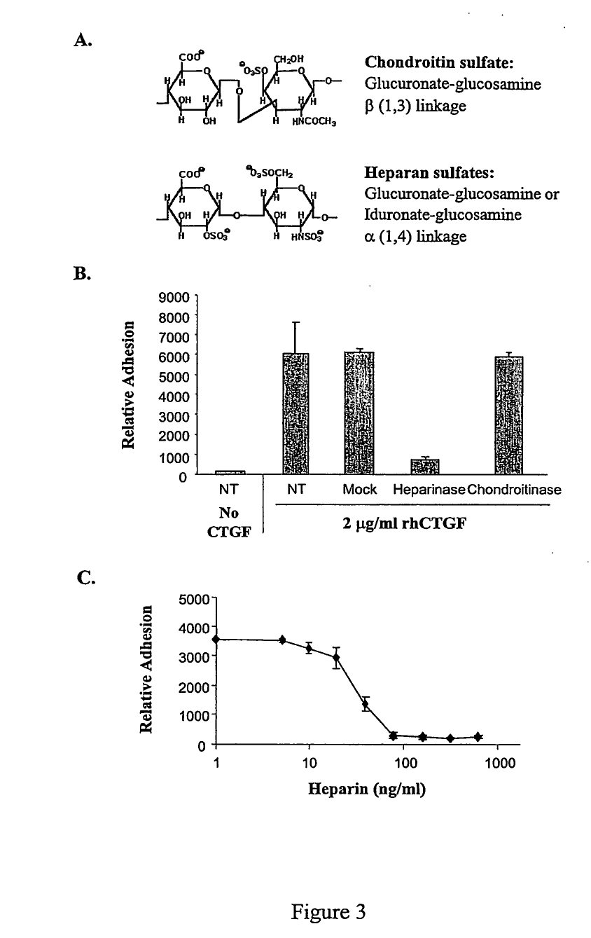

CTGF has been described as a heparin-binding growth factor. As cells may carry a variety of proteoglycan moieties on their surface, e.g., heparan sulfate, chondroitin sulfate, etc. (see FIG. 3A), the following experiment was conducted to determine the specificity of CTGF for such moieties. The cell adhesion and cell binding assays were conducted as described in Examples 3.1 and 3.2, respectively, except prior to seeding cells were treated for 1 hour at 37° C. with either vehicle, 4 units/ml heparinase I, or 2 units/ml chondroitinase ABC.

As shown in FIG. 3B, CTGF-dependent cell adhesion was inhibited by pretreatment of cells with heparinase, but not chondroitinase.

To further examine the requirement for heparan sulfate proteoglycans in CTGF-mediated cell adhesion, adhesion was measured in the presence of increasing amounts of heparin. Heparin and heparan sulphate both consist of repeating disaccharides of uronic acid and glucosamine, but the proportion of N-sulfation of heparan sulfate is typically below 50%, while sulfation of heparin is usually 70% or higher. The cell adhesion assay was conducted as described in Examples 3.1, except increasing concentrations of heparin was additionally added to each adhesion reaction.

As shown in FIG. 3C, CTGF-dependent adhesion was inhibited by soluble heparin in a concentration-dependent manner. This result supports the conclusion that CTGF-mediated cell adhesion requires heparan sulfate moieties, i.e., HSPGs.

### 5.2 Differential Inhibition of CTGF-Mediated Cell Adhesion and Cell Binding by Varying Sulfation of Polysaccharides

The sulfate groups of heparin include 2-O-sulfation of iduronate residues, 6-O-sulfation of iduronate residues, and amino group sulfation (N-sulfation) of glucosamine residues. Sulfates can be selectively removed using chemical methods known to those skilled in the art. Such methods, as described below, can be applied either solely or jointly to obtain a polysaccharide derivative with a desired sulfation pattern. Oligosaccharide libraries can be obtained and screened using methods known to those skilled in the art. (See, e.g., Jemth et al. (2003) J Biol Chem 278: 24371-24376; and Ashikari-Hada et al. (2004) J Biol Chem 10.1074/jbc.M313523200.)

Both O- and N-sulfate groups can be removed, e.g., by heating a pyridinium salt of heparin at 80° C. for four hours in dimethylsulfoxide. (See, e.g., Nagasawa et al. (1977) Carbohydr Res 58:47-55.) Since the elimination rate of the N-sulfate group is much greater than that of the O-sulfate group, carrying out the reaction under mild conditions, e.g., reaction at or below 20° C., produces selective de-N-sulfation. (See, e.g., Inoue and Nagasawa (1976) Carbohydr Res 46:87-95.)

Sulfate groups can be removed from ether (O-sulfation) linkages under strongly alkaline conditions. The resulting epoxide rings can then be cleaved to yield primarily iduronate residues. Removal of 6-O-sulfation can be carried out, e.g., as described in Takano et al. (1998, Carbohydr Lett 3:71-77).

To determine the specificity of sulfation and charge distribution for CTGF-mediated cell adhesion and cell binding, experiments as described in Examples 3.1, 3.2, and 3.3, were performed with the following modification. Combination of HFF cells with CTGF was accompanied by addition of increasing concentrations of heparin that was modified to contain differing amounts of sulfation and/or acetylation.

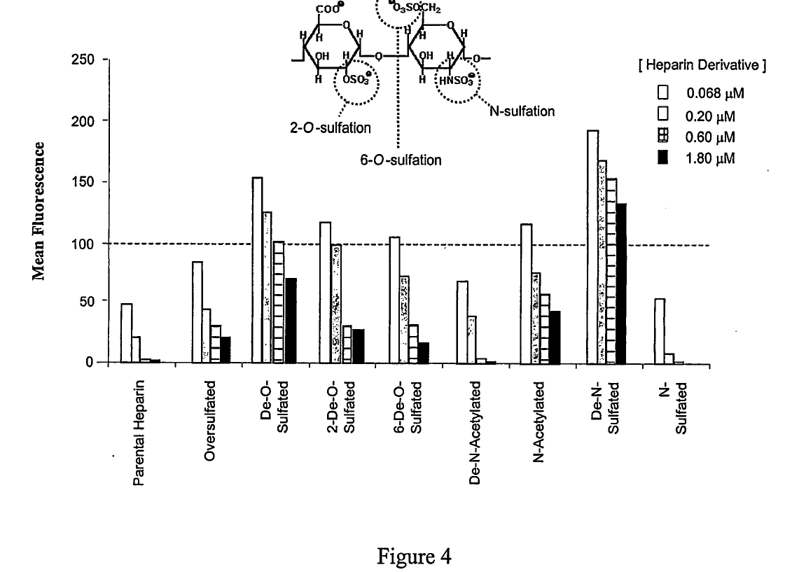

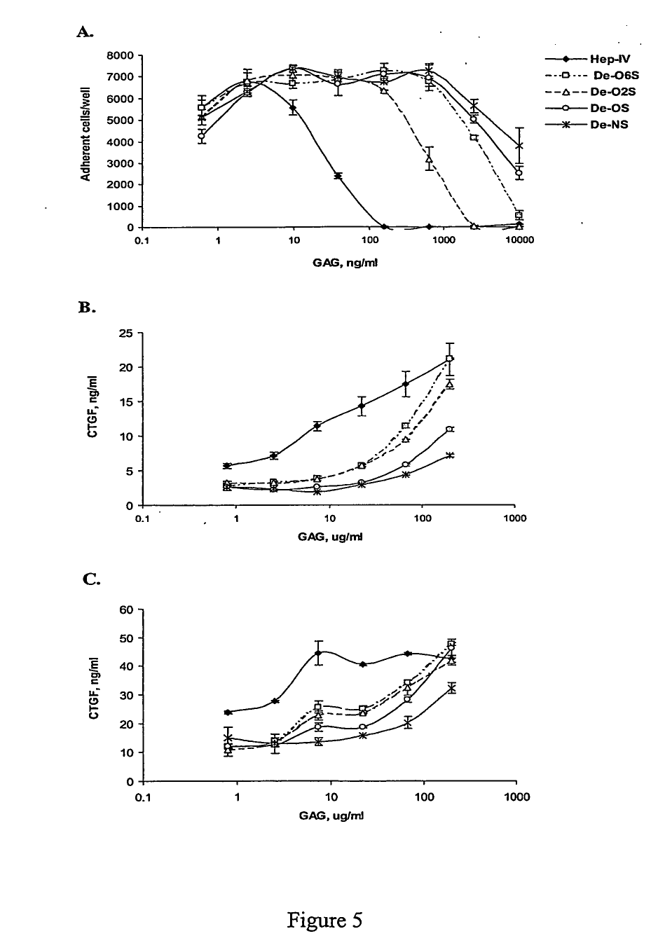

As shown in FIG. 4, binding of CTGF to HFF requires specific sulfation of heparan sulfate or heparin-like molecules. Specifically, heparin and oversulfated derivatives thereof substantially inhibit CTGF binding to cells. However, de-O-sulfated heparin derivatives were less effective at inhibiting binding, and de-N-sulfation showed no inhibitory capacity. Thus, cell binding by CTGF requires N-sulfation, and is further augmented by both 2-O- and 6-O-sulfation. The dashed line in FIG. 4 indicates the level of CTGF binding without any addition of heparin or derivatives. FIGS. 5A, 5B, and 5C, which show the effect of heparin or modified heparin on CTGF-mediated cell adhesion (FIG. 5A) and binding of CTGF to cells (HLF, FIG. 5B; and MG63, FIG. 5C), confirm the effect of desulfation seen in the CTGF binding assay above.

The data show that there are specific modifications on heparin sulfate that are critical for CTGF binding and cell adhesion, whereas other modifications do not affect CTGF binding or responsiveness. Specifically, the data point to the importance of N-sulfation and O-sulfation of sulfated polysaccharides as being critical for CTGF binding and signaling. These modifications are unique to CTGF and different from modifications known to mediate signaling of other heparin binding growth factors, such as, e.g., bFGF or PDGF. Thus, specific therapeutics can be derived based on heparan sulfate or heparin-like molecules which specifically inhibit CTGF function but do not inhibit the bioactivity of other heparin binding growth factors.

### 5.3 Differential Inhibition of CTGF-Mediated Cell Adhesion and Cell Binding by Sulfated Polysaccharides of Varying Size

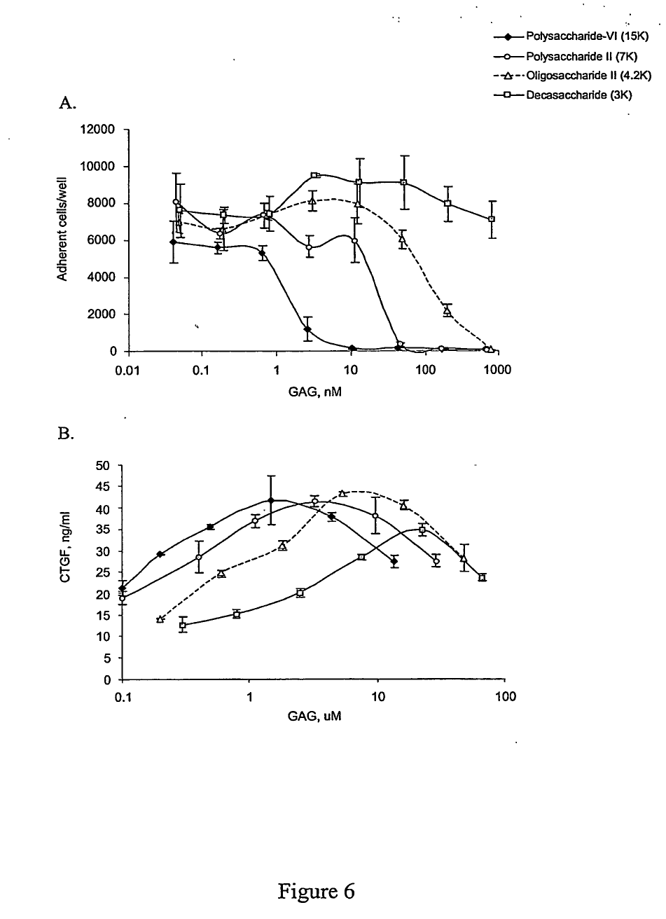

To determine size requirements for modulation of CTGF-mediated cell adhesion and cell binding, heparin moieties containing differing saccharide subunit number were examined. Polysaccharide lengths tested ranged from 10 to approximately 50 saccharides, and experiments were carried out as described in examples 3.1 and 3.3. As can be seen in FIG. 6, an oligosaccharide of approximately 14 saccharide subunits (4.2K) inhibited both CTGF-mediated cell adhesion (FIG. 6A) and binding of CTGF to cells (FIG. 6B). While a decasaccaride showed clear modulation of CTGF binding to cells, as shown in the displacement assay (FIG. 6B), it did not measurably affect CTGF-mediated cell adhesion.

The data show that a 10 saccharide moiety is capable of displacing CTGF from cells, thereby modulating CTGF interaction and signaling. Although CTGF-mediated adhesion appears to require a modestly longer polysaccharide for modulation, the increased length requirements may be due to additional interactions necessary for adhesion, e.g, interaction with integrins. (See, e.g., Gao and Brigstock (2003) J Biol Chem 10.1074/jbc.M313204200.) Clearly, a polysaccharide of at least approximately 14 saccharides (4.2K) is modulatory for CTGF-mediated cell adhesion and binding of CTGF to cells using the assays provided herein.

### 5.4 Differential Inhibition of CTGF-Mediated Cell Adhesion and Cell Binding by Sulfated Polysaccharides of Varying Saccharide Composition

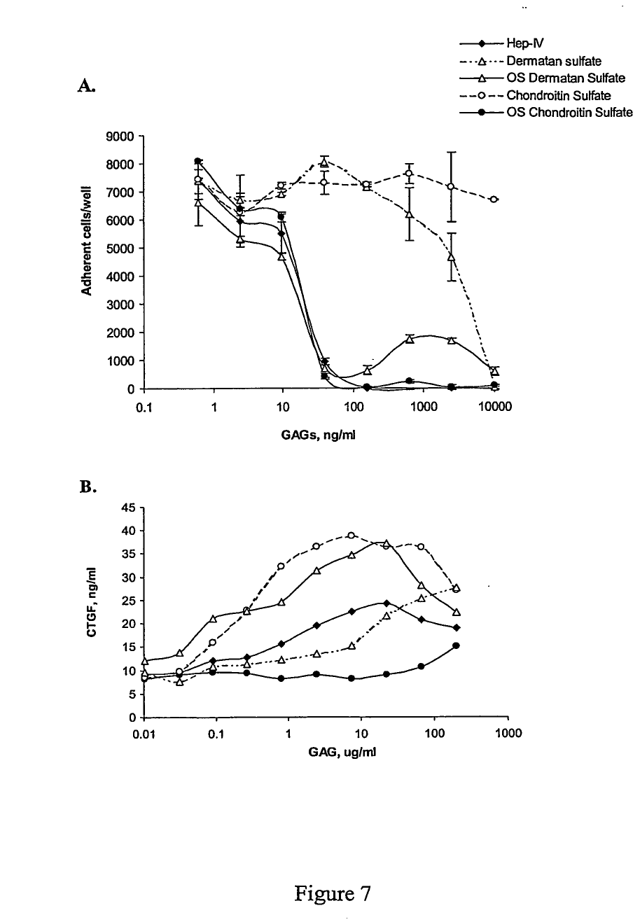

To determine flexibility in the saccharide composition of the compounds and agents of the invention, CTGF-mediated cell adhesion and binding of CTGF to cells was examined using various GAG constructs. As sulfation of the polysaccharide is clearly of importance to modulation of CTGF activities, the various GAGs were examined both in their natural sulfation state and in over sulfated (OS) constructs. (See Table 1.) Experiments were carried out as described in example 3.1 and 3.3. As can be seen in FIG. 7, naturally sulfated dermatan and chondroitin polysaccharides show no modulation of CTGF-mediated adhesion (FIG. 7A) or binding of CTGF to cells (FIG. 7B) except at high concentration. However, when sulfation of these polysaccharides is increased, both OS-chondroitin sulfate and OS-dermatan sulfate show substantial modulatory activity in both assays.

The data show that various polysaccharide backbones, including dermatan, and chondroitin, are capable of modulating CTGF-mediated cell adhesion and binding of CTGF to cells once the degree of sulfation is appropriate. Although neither naturally sulfated chondroitin or dermatan are capable of modulating CTGF activities, over sulfated constructs are equivalent to heparin in modulatory activity. Thus, various sulfated polysaccharides having appropriate length and sulfation density are useful for modulating CTGF activities, including CTGF-mediated cell adhesion and binding of CTGF to cells.

### Example 6

### Betaglycan is a CTGF-Binding HSPG

### 6.1 CTGF Binds Directly to Betaglycan

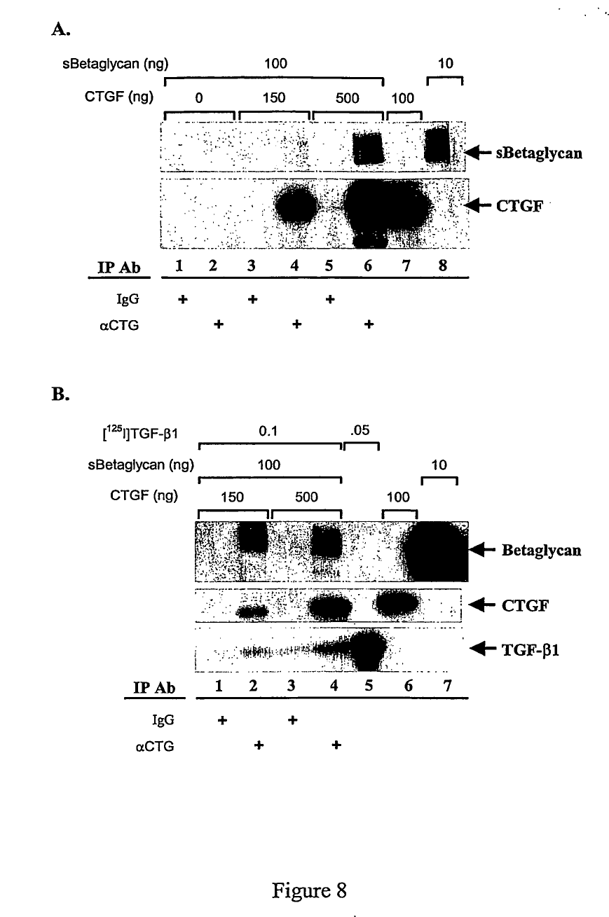

Identification of cell receptors for CTGF was carried out using co-immunoprecipitation procedures as described in Example 3.4. Initial experiments using radiolabeled cells identified betaglycan as a primary CTGF-binding protein on the cell surface (data not shown). Subsequent experiments using soluble betaglycan (sBetaglycan) demonstrated dose-sensitive interaction between betaglycan and CTGF (FIG. 8A). Together, the data show that betaglycan is a cell surface HSPG that functions as a specific receptor for CTGF.

### 6.2 CTGF Binds TGFβ and Betaglycan in a Ternary Complex in an HSPG-Dependent Fashion

Betaglycan is also known as TGF-β type III receptor and has been shown to facilitate cell stimulation by TGF-β. CTGF has also been associated with TGF-β signaling as an immediate early response factor produced by cells upon TGF-β signaling. To determine the functional nature of possible interactions between betaglycan, CTGF, and TGF-β, immunoprecipitations were performed as follows. Soluble betaglycan, [¹²⁵I]-labeled TGF-β, and CTGF were mixed under conditions suitable for interaction, and then complexes were isolated using anti-CTGF antibodies bound to a solid bead matrix. The data show that CTGF, betaglycan and TGF-β form a ternary complex that is dependent on the heparin binding potential of CTGF (FIG. 8B). The present invention contemplates that inhibition of ternary complex formation may inhibit betaglycan-dependent CTGF signaling, and may thereby modify TGF-β signaling.

### 6.3 CTGF Binds FGF and Betaglycan in a Ternary Complex in an HSPG Dependent Fashion

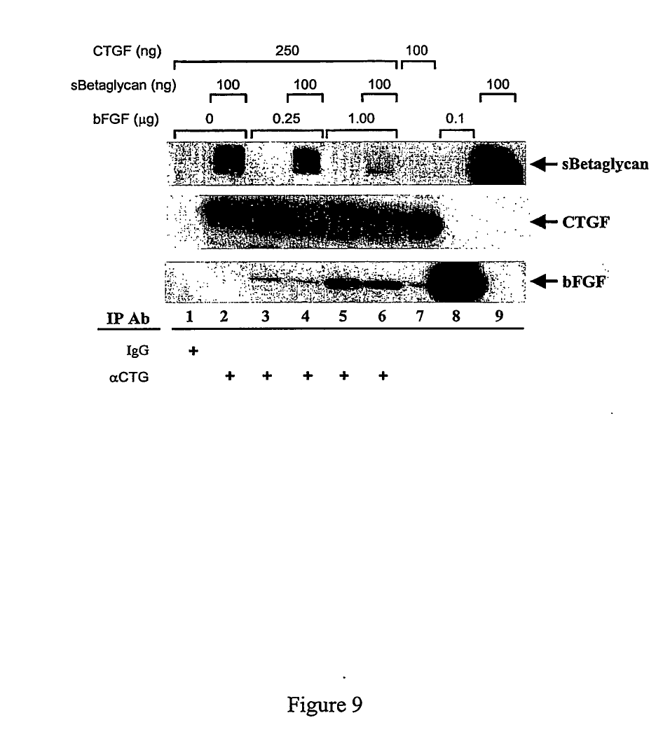

Fibroblast growth factors bind to HSPGs, and signaling by basic and acidic FGF requires this interaction. To determine if the HSPG-dependent interaction between CTGF and betaglycan involves or is modified by FGF, immuno-precipitations were performed as follows. Soluble betaglycan, bFGF, and CTGF were mixed under conditions suitable for interaction, and then complexes were isolated using anti-CTGF antibodies bound to a solid bead matrix. As shown in FIG. 9, binding between CTGF and betaglycan is adversely influenced by bFGF in a dose-sensitive manner. Surprisingly, the interaction was not due solely to competition between CTGF and bFGF to heparan sulfate moieties on betaglycan. There was also a clear interaction between CTGF and bFGF, as immunoprecipitation of CTGF in the presence of bFGF, without betaglycan, demonstrated clear interaction between the two growth factors. The result shows that a novel interaction between CTGF and bFGF has been identified, and that selective inhibition of ternary complex formation may inhibit CTGF signaling alone, coordinated signaling between CTGF and TGF-β, and/or coordinated or independent signaling by bFGF.

Various modifications of the invention, in addition to those shown and described herein, will become apparent to those skilled in the art from the foregoing description. Such modifications are intended to fall within the scope of the appended claims.

All references cited herein are hereby incorporated by reference in their entirety.

1. A compound or agent for modulating CTGF-mediated cell adhesion.

2. A compound or agent for modulating binding of CTGF to a cell.

3. The compound or agent of claim 1, wherein the CTGF is directly adsorbed to the substrate.

4. The compound or agent of claim 1, wherein the CTGF is bound to a monoclonal antibody specific for a CTGF.

5. The compound of claim 4, wherein the antibody binds to an epitope contained within a region of human CTGF from amino acid 1 to 247 or to an orthologous region of a CTGF from another species, and wherein the antibody is adsorbed to the substrate.

6. The compound or agent of 1, wherein CTGF is selected from the group consisting of endogenous CTGF, recombinant CTGF, and fragments of CTGF.

7. The compound or agent of claim 6, wherein the fragments of CTGF comprise at least amino acid 247 to 349 of human CTGF or an orthologous region of a CTGF from another species.

8. The compound or agent of claim 1, wherein the cell is selected from the group consisting of a fibroblast, an endothelial cell, and an osteosarcoma cell.

9. The compound or agent of claim 1, wherein the compound or agent is a sulfated polysaccharide.

10. The compound or agent of claim 9, wherein the polysaccharide comprises at least 10 saccharide subunits.

11. The compound or agent of claim 9, wherein the polysaccharide comprises about 10 to 50 saccharide subunits.

12. The compound or agent of claim 9, wherein the polysaccharide comprises a repeating disaccharide, wherein one saccharide substituent is selected from the group consisting of N-galactosamine and N-glucosamine, and the other saccharide substituent is selected from the group consisting of iduronate, glucuronate, and galactose.

13. The compound or agent of claim 12, wherein the polysaccharide is selected from the group consisting of dermatan, chondroitin, and heparan.

14. The compound or agent of claim 9, wherein the polysaccharide contains at least 1.5 sulfate groups per disaccharide.

15. The compound or agent of claim 9, wherein the polysaccharide contains at least 2.0 sulfate groups per disaccharide.

16. The compound or agent of claim 9, wherein the polysaccharide contains about 2.0 to 3.5 sulfate groups per disaccharide.

17. Use of a compound or agent of claim 1 to modulate CTGF-mediated cell adhesion in a subject.

18. Use of a compound or agent of claim 2 to modulate binding of CTGF to a cell in a subject.

19. The use of claim 18, wherein the subject is selected from a cell, a tissue, and an organ, and the use is performed ex vivo.

20. The use of claim 18, wherein the subject is a mammal.

21. The use of any one of claim 20, wherein the subject is a human.

22. The use of claim 18, wherein the subject has or is at risk for having a CTGF-associated condition or disorder.

23. The use of claim 22, wherein the CTGF-associated condition or disorder is selected from the group consisting of fibrosis, metaplasia, and cancer.

24. The use of claim 22, wherein the CTGF-associated condition or disorder is idiopathic pulmonary fibrosis.

25. The use of claim 22, wherein the CTGF-associated condition or disorder is diabetic nephropathy.

26. A method for identifying compounds or agents that modulate CTGF-mediated cell adhesion, the method comprising:
a) adsorbing a monoclonal antibody specific for CTGF to a first and second substrate;
b) binding CTGF to the antibody on the first and second substrate;
c) adding cells to the first substrate under suitable conditions for cells to adhere to CTGF;
d) adding a compound or agent and cells to the second substrate under suitable conditions for cells to adhere to CTGF; and
e) comparing the number of cells adhered to CTGF on the first substrate and the number of cells adhered to CTGF on the second substrate, wherein a difference between the number of cells adhered to the first substrate compared to the second substrate is indicative of a compound or agent that modulates CTGF-mediated adhesion.

27. The method of claim 26, wherein the monoclonal antibody binds to a CTGF epitope contained within a region of human CTGF from amino acid 1 to 247 or to an orthologous region of a CTGF from another species, and wherein the antibody is adsorbed to the substrate.

28. A method for identifying compounds or agents that modulate binding of CTGF to a cell, the method comprising:
f) culturing cells capable of producing endogenous CTGF in the presence of a compound or agent for a suitable period of time;
g) measuring the level of CTGF in the culture medium; and
h) comparing the amount of CTGF in the culture medium to the amount of CTGF in culture medium from cells cultured in the absence of compound for an identical period of time, wherein a difference between the amount of CTGF in culture media in the presence of compound or agent relative to in the absence of compound or agent is indicative of a compound or agent that modulates binding of CTGF to a cell.

29. The method of any one of claim 28, wherein the cell is selected from the group consisting of a fibroblast, an endothelial cell, and an osteosarcoma cell.

30. A method for identifying compounds or agents that modulate interaction between CTGF and an HSPG, the method comprising:
i) incubating CTGF and the HSPG in the presence of a compound or agent under conditions suitable for interaction between CTGF and the HSPG;
j) measuring the amount of HSPG interacting with CTGF; and
k) comparing the amount of HSPG interacting with CTGF in the presence of compound to the amount of HSPG interacting with CTGF in the absence of compound, wherein a difference between the amount of HSPG interacting with CTGF in the presence of compound or agent relative to in the absence of compound or agent is indicative of a compound or agent that modulates interaction between CTGF and the HSPG.

31. The method of 30, wherein CTGF is selected from the group consisting of endogenous CTGF, recombinant CTGF, and fragments of CTGF.

32. The method of claim 31, wherein the fragments of CTGF comprise at least amino acid 247 to 349 of human CTGF or an orthologous region of a CTGF from another species.

33. The method of claim 30, wherein the HSPG is selected from the group consisting of betaglycan and LDL receptor-related protein (LRP).
